(19) United States
(12) Patent Application Publication
(10) Pub. No.: US 2008/0154245 A1
(43) Pub. Date: Jun. 26, 2008

(54) Nano materials application in durable medical equipment and method of using same

(76) Inventor: James Jay Martin, Oklahoma City, OK (US)

Correspondence Address: Martin G. Ozinga;Phillips McFall McCaffrey McVay & Murrah, P.C., Corporate Tower, 13th Floor, 101 N. Robinson Ave., Oklahoma City, OK, 73102, US

(21) Appl. No.: 12/002,879

(22) Filed: Dec. 19, 2007

Related U.S. Application Data
(60) Provisional application No. 60/876,021, filed on Dec. 20, 2006.

(51) Int. Cl. A61B 17/00

(52) U.S. Cl. 606/1; 977/931

(57) ABSTRACT

It is contemplated that the current invention may comprise applications utilizing nano-materials, such as but not limited to, superhydrophobic nano-materials, or carbon based nano-materials which may be applied in a spray, wipe on, powder, manufactured into the material or surface of durable medical equipment, devices, prosthetics and so forth.

### CROSS-REFERENCE TO RELATED APPLICATIONS

Priority is claimed from provisional U.S. patent application Ser. No. 60/876,021 filed on Dec. 20, 2006, and incorporated by reference herein.

### BACKGROUND OF THE INVENTION

1. Field of the Invention

In general, the present invention relates to nano-materials for durable medical equipment. More particularly, the present invention relates to a new and improved application of nano-materials to provide a water repellant surface, transfer heat, and provide more hygienic conditions for medical equipment, devices, prosthetics, and so forth.

2. Description of the Prior Art

In the fields of prosthetics, orthotics, rehabilitative care, and hospital/medical care, and durable medical equipment in general, a great need exists for increased hygiene, cleanliness, and use related durability. These devices are often subject to human contact in environments where hygiene is of great concern.

In recent years, advancements in nano-materials have provided technologies to enhance products in numerous fields. These nano-technologies, including, but not limited to carbon nano-tubes and superhydrophobic surfaces, stand to greatly benefit the medical community in their ability to promote hygiene and function of medical devices.

While much consideration has been given to their application in implantable devices such as dental and implantable joint replacements, further consideration is needed in rehabilitative care and other durable medical equipment.

The inclusion of developments in nano-materials such as superhydrophobic surface coatings may help alleviate some of the issues surrounding the use of durable medical equipment. These nano-materials that have been developed through others' efforts stand to find applications in new arenas, enhancing products in fields that have not yet been applied to.

A superhydrophobic surface is a hydrophobic material further enhanced by surface nanostructure. Energetically, on a molecular level, water prefers to bind to itself instead of the superhydrophobic coated surface so strongly that a thin layer of air literally separates most of the contact interface between the water and superhydrophobic surfaces.

The lotus leaf is a natural form of a superhydrophobic surface. A considerable amount of research is taking place currently to better replicate the characteristics of the lotus leaf. As these newer and more robust methods are developed, the application of these technologies will find additional applications in various fields.

For many flat solid materials, the interaction of water and the material can be classified as either hydrophilic or hydrophobic. The contact angle of the drop of water with the surface quantifiably determines the hydrophilic or hydrophobic nature of that surface. The contact angle is the interior angle between the material surface and the water drop surface.

If the contact angle that the water drop meets the surface is less than 90 degrees, the material is hydrophilic and indicates that the water tends to bind to the surface more than itself. If the contact angle is more than 90 degrees, the material is classified as hydrophobic and indicates that the water prefers to bind to itself more than the surface.

If a material has a larger water drop contact angle than 150 degrees, the surface is classified as superhydrophobic. To create a superhydrophobic surface, micro or nano-scale surface structures must be present to amplify the hydrophobic nature of the surface. These surfaces actually allow water to roll off, leaving the surface completely dry—a phenomena known as de-wetting.

Because the surface so well repels water, the surface is actually never wet. Spraying water on the surface may actually collect dust, bacteria, and other residue laying on the surface, and then immediately run off, leaving the surface cleaner than would be possible with washing it alone on a non superhydrophobic surface. This unique behavior is observed with distilled water, rain water, tap water, ocean salt water, and water with bacteria or algae in it.

The current industry is constantly looking for improved means to repel water, provide hygienic conditions, and reduce complications associated with unwanted water and fluid in general. Thus, there is a need for a new and improved construction of same. The current invention provides a superior application where the prior art fails.

### SUMMARY OF THE INVENTION

In view of the foregoing disadvantages inherent presently in the prior art, the current invention provides a viable solution and alternative. As such, the general purpose of the current invention, which will be described subsequently in greater detail, is to provide a new and improved water repellant application utilizing nano-materials which has all the advantages of the prior art and none of the disadvantages.

It is contemplated that the current invention may comprise applications utilizing nano-materials, such as but not limited to, superhydrophobic nano-materials, or carbon based nano-materials which may be applied in a spray, wipe on, powder, manufactured into the material or surface and so forth. It is understood that with some nano-material technologies, and surface material properties, the benefits of the nano-material surface coating may wear off over time. Through using a solution that can be re-applied to reinstate the nano-material properties, the material surface may remain as desired.

Hygienic or superhydrophobic layers may further comprise at least one of a vapor deposited, sprayed, filmed, painted, coated, applied as a solvent, melted or other forms of deposition layer. Additionally, the carbon or non-carbon based nano-material may be applied through chemical vapor deposition or laser vaporization to form a highly thermally conductive nano-material or nano-composite, among other methods.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception upon which this disclosure is based may readily be utilized as a basis for the designing of other structures, methods, and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Therefore, it is an object of the present invention to provide a new and improved nano-material application and method of using the same for durable medical equipment to provide a cleaner surface for contact with human use.

It is a further object of the present invention to provide a new and improved nano-material application and method of using the same which is of a durable and reliable construction and may be easily and efficiently manufactured and marketed.

An even further object of the present invention is to provide a new and improved nano-material application and method of using the same which is susceptible to a low cost of construction with regard to both materials and labor, and which accordingly is then susceptible to low prices of sale to the consuming public, thereby making such invention economically available to those in the field and public in general.

Still another object of the present invention is to provide a new and improved nano-material application and method of using the same where the prior art fails and provides all of the advantages of the prior art, while simultaneously overcoming some of the disadvantages normally associated therewith.

While still another object of the present invention is to provide a new and improved nano-material application and method of using the same which allows for utilization in new equipment, devices and so forth.

It is a further object of the present invention to provide a new and improved nano-material application and method of using the same which allows for utilization in existing equipment, devices and so forth by means of retrofitting or application to same.

Furthermore, it is a further object of the present invention to provide a new and improved nano-material application and method of using the same that allows for the heat transfer, removal or diminishing of heat from a user.

Still furthermore, it is a further object of the present invention to provide a new and improved nano-material application and method of using the same that may be utilized to enhance durable medical equipment and other rehabilitative, medical, or human contact surfaces.

Yet another object of the present invention is to provide a new and improved nano-material application and method of using the same that provides user friendly application as well as diminishes the hazards associated with non-hygienic conditions associated with medical equipment and devices in general.

Still another object of the present invention is to provide a healthy environment for medical patients as well as medical care givers thereby decreasing the risk associated with bacterial and viral transfers.

These, together with other objects of the invention, along with the various features of novelty which characterize the invention, are pointed out with particularity in the claims annexed to and forming a part of this disclosure. For a better understanding of the invention, its operating advantages, and the specific objects attained by its uses, reference should be had to the accompanying drawings and descriptive matter in which there are illustrated preferred embodiments of the invention.

The present invention referred to throughout may be embodied in other specific forms without departing from its spirit or essential characteristics. The described embodiments are to be considered in all respects only as illustrative and not restrictive. Furthermore, each of the methods that have been described should also be considered only as illustrative and not restrictive.

### BRIEF DESCRIPTION OF THE PICTORIAL ILLUSTRATIONS GRAPHS, DRAWINGS, AND APPENDICES

The invention will be better understood and objects other than those set forth above will become apparent when consideration is given to the following detailed description thereof. Such description makes reference to the annexed pictorial illustrations, graphs, drawings, exhibits and appendices wherein:

FIG. 1 is a general schematic defining the contact angles of a water drop on a surface. This figure as well demonstrates a hydrophobic natured water drop against the surface.

FIG. 2A is a hydrophilic surface demonstrating a contact angle less than 90 degrees.

FIG. 2B is a superhydrophobic surface demonstrating a contact angle greater than 150 degrees.

FIG. 3 is a lateral posterior view of a prosthetic transtibial gel liner, with superhydrophobic nano-material coating on it in accordance with a preferred embodiment of the invention.

FIG. 4 is a general depiction of a water drop traversing across a surface coated with superhydrophobic nano-technology, and its ability to carry off debris from the surface in accordance with a preferred embodiment of the invention.

FIG. 5 is a general view of a preferred embodiment of the invention wherein nano-technology application in human contact DME devices, specifically a wheelchair in this particular example, and its ability to transfer heat away from the body.

### DETAILED DESCRIPTION OF THE PREFERRED EMBODIMENTS

This disclosure, in general, describes a new and improved application of nano-materials to enhance durable medical equipment (DME) and other rehabilitative, medical, or human contact surfaces. Items including, but not limited to, hospital equipment, beds, wheelchairs, external prosthetics devices such as artificial limbs, orthotics, crutches, and other such rehabilitation and patient care equipment suffers from frequent patient contact, and degradation of the DME due to wear and hygiene issues.

These nano-materials that may be applied may encompass methods such as, but not limited to, ordered or non ordered arrays of spikes or other uniform or non uniform shapes coated or not coated with a self assembled monolayer or multi-layer of other hydrophobic materials. The “spikes” may be made of glass, polymers, films, porous or non porous microspheres or nanofibers composite film via electrohydrodynamics or not, PNFT coated material, polyelectrolyte multilayers or other materials, and may be bonded with adhesives or other methods of implementing within manufactured processes. It is understood that there are many other illustrations of providing a superhydrophobic surface that may be implemented as well, and the descriptions included should in no way be considered limiting.

Other nano-materials such as carbon nano-tubes, string-like structures formed of carbon atoms bonded in a helix or other patterns, may be applied as well to promote the effects of the superhydrophobic materials. Carbon, one of the strongest naturally occurring substances, provides a sturdy framework for material durability and stands to improve the durability of other materials and structures. The integration of carbon nano-tubes along with other nano-based structures, such as superhydrophobic nano-materials, stands to enhance the benefits of both—providing a surface that has increased strength from the carbon nano-tubes and has superhydrophobic properties. The superhydrophobic surface nano-materials may provide a more protected environment for the volatile surface of the carbon nano-tubes along the material, while the carbon nano-tubes will help to strengthen the bonding of the superhydrophobic materials to the surface and not wear off over time.

The nano-materials, such as but not limited to, superhydrophobic nano-materials, or carbon based nano-materials may be applied in a spray, wipe on, powder, or other forms such as but not limited to being manufactured into the material or surface. It is understood that with some nano-material technologies, and surface material properties, the benefits of the nano-material surface coating may wear off over time. Through using a solution that can be re-applied to reinstate the nano-material properties, the material surface may remain as desired.

Hygienic or superhydrophobic layers may further comprise at least one of a vapor deposited, sprayed, filmed, painted, coated, applied as a solvent, melted or other forms of deposition layer. Additionally, the carbon or non-carbon based nano-material may be applied through chemical vapor deposition or laser vaporization to form a highly thermally conductive nano-material or nano-composite, among other methods.

This superhydrophobic nano-technology may begin in the raw form of an application powder or other substance forms, and may be applied through many different means to coat the desired surface. For simplification purposes, it will be referred to as an application powder or powder, but that term should in no way be considered limiting, as it may be applied through various known methods. This powder may then be applied to the surface of a material.

At a microscopic level, surfaces are uneven, and show rivets on their edges. This powder, when applied, forms an additional layer of prominences such as but not limited to cones or other regular or irregular shapes that are packed tightly together across this uneven surface. Because of their small distance apart, particles of water, dirt and other molecules will sit on top of the microscopic shaped structured material, creating a protective barrier above the actual surface. This barrier does not allow for adhesion or penetration to the surface of various other substances, thus allowing foreign particles to be easily washed away.

This application powder can be coated onto most any surface, made of most any solid or flexible material. The result is a material surface that is water-repellent. Consequently, the surface has properties in which loosely bound dirt particles will be picked up by water to create a hygienic cleaning effect on the surface, and then will immediately be dry once the water runs off. Since the water has incredibly low surface tension, it will tend to run off of the material, and provide a structure for dirt, bacteria, and other debris to stick to rather than the material surface such as the DME device.

The use of such nano-materials on DME devices would be applicable to many surfaces used within the hospital, DME, rehabilitative care, orthotics, and prosthetic technologies, including cosmesis coverings, sockets, suspension sleeves, socks, and the gel liners used by the patient. All such prosthetic devices comprising a prosthetic system and in general human contact DME equipment, generally comprising nano-materials, and more specifically that may comprise superhydrophobic materials are claimed under this patent application.

Due to the nature of the superhydrophobic substance, its application to a prosthetic material, such as a liner, will cause sweat and odor build-up to be eliminated, providing a more hygienic environment for the sensitive residual limb.

Furthermore, the superhydrophobic nature will further ease in donning abilities due to the application powders' ultra slick surface. Cleaning the material will be less time constraining with nearly instantaneous drying of the material. And, the products structure will be improved with the lack of saturation of the material with sweat and other buildup, as well as through embedding of carbon nano-tubes or other structurally supporting materials, providing greater durability and strength.

Incorporating super hydrophobic nanotechnology onto prosthetics, orthotics, and durable medical equipment in general may be accomplished with any number of known methods in the art. In general, it is understood that the integration of super hydrophobic nanotechnology with prosthetics and orthotics, and durable medical equipment in general, offers unique advantages of those products over pre-existing prior art.

These methods of incorporating the super hydrophobic nanotechnology with the polymer substrate or other similar or dissimilar materials may include, but are not limited to the following preferred embodiments.

One known method of applying super hydrophobic nanotechnology to these surfaces may include using a glass substance that may be composed of sodium borate and silicate glass. Other known materials may be used as well though. This composition may be heat treated to separate the substances, which may create it to be opalescent in nature. This substance may then be ground up further into a fine powder, or may be sonicated or other known methods of reducing the particle size. The power may then be differentially etched to remove much or all of the sodium borate and possibly part of the silicate substance. This glass powder that is left may be amorphous silica that is porous. This may then be ground up further into sizes of one micron or smaller, though can be larger as well.

Applying phorinated silene or similar substances may then alter the surface chemistry of this new substance. This may actually be used to provide a thin layer of covalently bonded silocene, or similar, that may be only a few molecules thick. This may create the surface to be similar to Teflon in nature, creating the super hydrophobic qualities.

Additionally, this super hydrophobic nanotechnology powder may be bonded by flash melting it to the surface of the material (possibly a polymer substrate or other materials) with a solvent. Using a flash melting process onto the surface may create a mechanical bond to the surface adding additional integrity of the surface coating.

In another method of integrating the super hydrophobic nanotechnology to the surface material, a solvent may be used with a binder mixed in with the solvent, with the powder mixed in as well. This may then be painted, or similar, onto the material surface, allowing the bonding agent to bind to the substrate and the powder.

In another embodiment, the powder may not be pre-treated, and a binder may be used to adhere to the surface. The surface with the binder and powder may then be treated later to create the super hydrophobic nature.

It should be understood that the invention disclosed is for the application of these described nano-materials. The further descriptions of the make up of the nano-materials is for explanatory purposes, and should not be considered limiting. These explanations are used to describe the functions of the nano-materials and their properties as they relate to durable medical equipment and human contact surfaces.

The use of other non-superhydrophobic nano-technology based material may be applied to such DME, hospital, rehabilitation, and other such human contact surfaces, for purposes of enhancing cleaning abilities, minimizing bacteria, odor, infectious diseases, or other similar or complimentary purposes, may be applied.

Referring now to the drawings, wherein like reference numerals designate corresponding structure throughout the views, and referring in particular to FIGS. 1-5, reference numeral 100 generally refers to a new and improved nano-material application and method of using the same in accordance with the present.

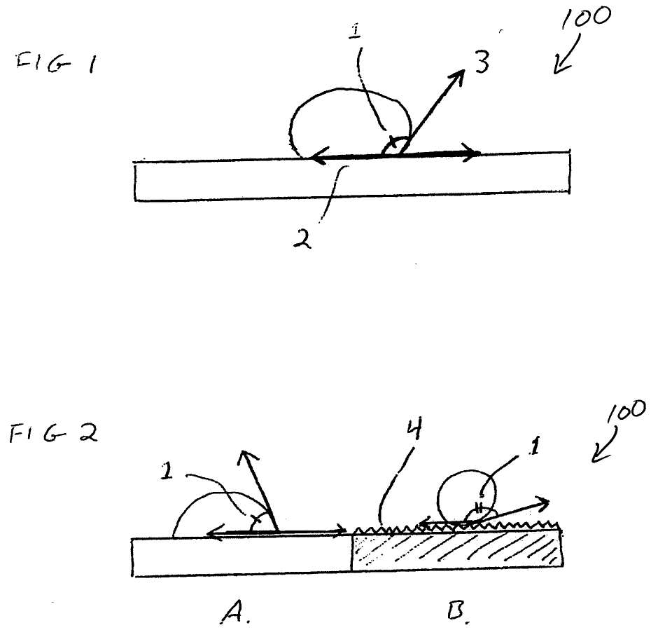

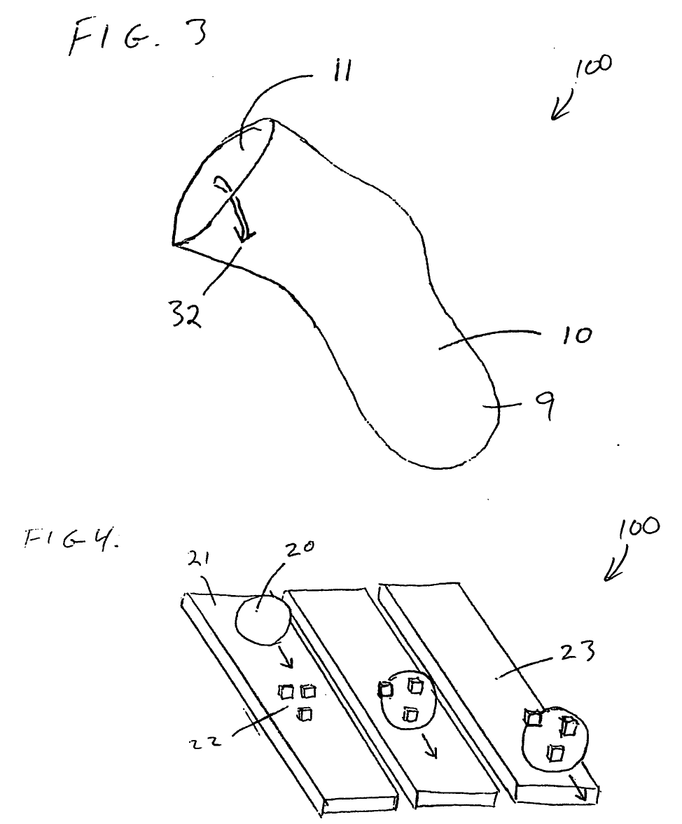

FIG. 1 is a general schematic defining the contact angles of a water drop on a surface. The contact angle is the interior angle 1 between the material surface 2 and the water drop surface 3. If the contact angle that the water drop meets the surface is less than 90 degrees (see FIG. 2A), the material is hydrophilic and indicates that the water tends to bind to the surface more than itself. If the contact angle is 90 degrees to 150 degrees (see FIG. 1), the material is classified as hydrophobic and indicates that the water prefers to bind to itself more than the surface.

If a material has a larger water drop contact angle than 150 degrees (see FIG. 2B), the surface is classified as superhydrophobic. To create a superhydrophobic surface, micro or nano-scale surface structures 4 must be present to amplify the hydrophobic nature of the surface. Additional coatings such as but not limited to Rain-X or similar hydrophobic coatings (not depicted in the figure) may be applied over the surface structures 4 to further enhance the superhydrophobic function of the surface structures 4.

FIG. 3 illustrates a prosthetic gel liner 9 coated with the superhydrophobic nano-material. The external or outer surface 10 of the liner 9 may have fabric on it to better assist in donning of the liner 9 and help increase durability. The inner surface 11 of the liner 9 typically is a gel-like material to be in contact with the skin. Both the inner 11 and outer 10 surfaces may be coated with the superhydrophobic nano-material to provide greater ease of donning (slicker surfaces), better hygiene (cleaner surfaces), and be better wear resistant in nature, among other benefits.

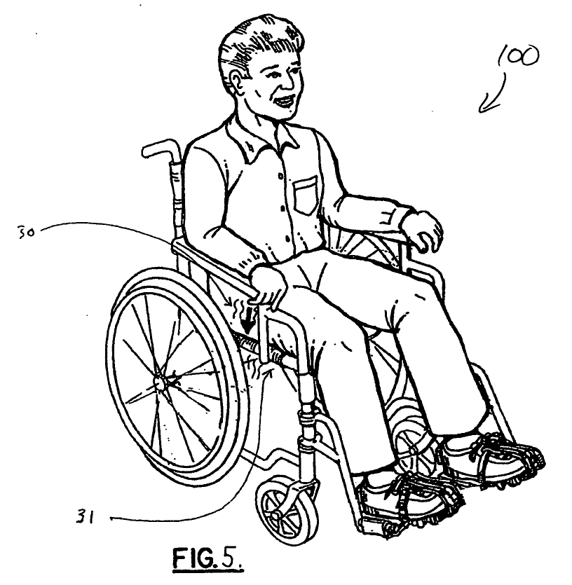

FIG. 4 illustrates a water drop 20 in contact with a superhydrophobic surface 21. The surface illustrated has debris 22 on it which is picked up by the water drop 20 upon contact. Because of the low surface tension with the superhydrophobic surface 21, the debris 22 tends to bond itself to the water drop 20 upon contact, leaving a cleaner surface 23.

While in illustration FIG. 3, a prosthetic gel liner is referenced, it is contemplated that similar benefits occur with other rehabilitation, hospital, and DME devices which are in contact with humans and other sources of infectious diseases, germs, debris, etc, and including other prosthetic components such as suspension sleeves, socks, sockets, cosmetic coverings and others.

Furthermore, it is contemplated the use of such nano-technologies may be applied to existing prosthetics and DME and other devices through a post-manufacture application. This application may be at the place of manufacture, or by a consumer. Furthermore, it may be manufactured in with the product at time of manufacture.

The utilization of this application in prosthetics, as example, helps to provide a skin-contact material that is easier to clean, stays cleaner than other conventional systems, does not have odors from sweat, lasts longer by the body fluids not breaking down the material, and having a better general appearance and be antimicrobial in nature. Additionally, the superhydrophobic characteristics on the outside of the liner, and other parts of the prosthesis will exhibit similar effects. It is contemplated that the superhydrophobic material will benefit other prosthetic components, which incidentally may use similar materials as the liner, such as but not limited to suspension sleeves, socks, sheaths, gel padding, sockets, cosmetic coverings, componentry, and other such prosthetics devices.

Still furthermore, the lubricous characteristics from superhydrophobic coatings may lead to ease donning of liner or other prosthetic human interface systems. Providing a novel system for facilitating the donning and wearing of a prosthesis, as well as use of other DME devices, is characterized by reducing the frictional forces between the residual limb and the prosthesis through using this nano-technology. The superhydrophobic characteristics inherently benefit these devices through alleviating one of the major troublesome issues surrounding their use in clinical considerations.

Furthermore, the superhydrophobic coating would make the cosmesis very water-repellent and could allow the cosmesis material to become more “skin like” in appearance and texture. In addition, because the superhydrophobic material effectively repels dirty water, it will likely stay cleaner (and be more hygienic) in wet environments and further help to protect the sensitive underlying components by de-wetting the surface and quickly keep the water away from seeping into small protrusions in the cosmetic covering. Additionally, the use of such nano-technology will stand to improve the tear strength of the prosthetics or other DME used materials, further protecting the device from unnecessary wear and tear.

Further use of the superhydrophobic application in prosthetics and other DME applications can be used for the protection of electrical equipment and underlying substructures. Since water and other debris will no longer be adhering to the surface, the volatile and sensitive electronics generally associated with many of the newer technologies in rehabilitative care will be protected.

The use of superhydrophobic and/or carbon based technology to protect MEMS based devices is claimed under this patent application. MEMS technology stands to provide great solutions for prosthetics and DME applications but remains volatile in its application within such devices. The use of carbon nano-tubes and/or superhydrophobic technology may provide a viable solution for the practical application of MEMS within such devices.

Additionally, the use of this technology can be subsequently applied to orthotic devices, as many of the same materials are used in orthotics as are used in prosthetics.

The use of superhydrophobic nano-materials in association with human contact and DME devices to promote hygiene, cleanliness, durability, and the like, is claimed under this invention.

Still furthermore, it is contemplated that the current invention may use nano-technology, and in particular, but not limited to, the use of carbon nano-technology, be applied to the wearable components of the prosthesis or other body contact DME devices to assist in cooling. Carbon nano-technology, as well as other nano-technologies, offers significant heat transfer characteristics which stand to benefit such human contact devices, as generally depicted in FIG. 5. Heat is transferred from the user's body 30, through the cushioning device 31, as depicted in this case, through using nano-technology embedded within the human contact device 31. This example should not be considered limiting, and is used for explanatory purposes. It should be understood that similar and complimentary benefits will be observed through using other devices such as but not limited to prosthetic sockets, liners, and sleeves, orthotics, beds, rehabilitation equipment, and the like. It should be understood that this surface may as well incorporate and benefit from the application of superhydrophobic nano-materials.

Additionally, as further illustrated in FIG. 3, heat transfer 32 is illustrated as going from the user's residual limb, through the nano-based material in the prosthetic interface system, to the outside environment through a similar means as described in FIG. 5.

In prosthetics in particular, as well as what would be found in other DME devices and the like, a sense that the device is excessively hot provides an uncomfortable environment for the user. Utilizing such technologies to cool the perceived environment for amputees, users of orthotics, bed ridden victims, wheel chair users, and the like, will significantly benefit quality of life for those users. The incorporation of such nano-technologies to assist in cooling characteristics or DME, rehabilitation, and hospital-type devices is claimed under this patent application.

While there may be a risk of cytotoxicity and other ailments from carbon or non-carbon based nano-materials in contact with the skin, it is understood that utilizing a coating between the nano-materials and the human skin may be applied to mitigate these effects.

While the invention has been described with a certain degree of particularity, it is manifest that many changes may be made in the details of the construction and the arrangements of the components and method of implementation without departing from the spirit and scope of this disclosure. It is understood that the invention is not limited to the embodiments and methodologies set forth herein for purposes of exemplification, but is to be limited only by the scope of the explanations of the invention, including the full range of equivalency to which each element thereof is entitled. Changes may be made in the combinations, operations, and arrangements of the various parts and elements described herein without departing from the spirit and scope of the invention.

I claim:

1. A method for using nano-materials as described above.

2. A system utilizing nano-materials as described above.

3. A nano-material application as described above.
